(19) United States
(12) Patent Application Publication
(10) Pub. No.: US 2025/0285764 A1
(43) Pub. Date: Sep. 11, 2025

(54) METHODS AND SYSTEMS FOR DETERMINING AN INTERVENTION BASED TREATMENT FOR CHRONIC MEDICAL CONDITIONS

(71) Applicant: Sequelae, Inc., Minneapolis, MN (US)

(72) Inventors: Sara Taylor, Edina, MN (US); Mark Briesacher, Park City, UT (US); Scott Archibald, Wayzata, MN (US); Mike Christy, Shakopee, MN (US); Steven J. Catani, Athens, GA (US)

(73) Assignee: Sequelae, Inc., Minneapolis, MN (US)

(21) Appl. No.: 19/072,084

(22) Filed: Mar. 6, 2025

Related U.S. Application Data
(60) Provisional application No. 63/562,908, filed on Mar. 8, 2024.

(51) Int. Cl. G16H 50/30; A61B 5/00; A61B 10/00; G16H 10/40; G16H 20/10; G16H 20/70; G16H 50/70

(52) U.S. Cl. CPC G16H 50/30; A61B 5/4842; A61B 10/0045; G16H 10/40; G16H 20/10; G16H 20/70; G16H 50/70; A61B 2010/0003

(57) ABSTRACT

Methods and systems for intervention-based treatment for chronic medical conditions are described. In one method, a member of a population is identified as having a disease by identifying an increased level of a biological marker. If the member's levels are elevated or increasing, the method provides an intervention for the member based on the level of the biological marker. For example, the intervention may be a pharmaceutical or lifestyle intervention (e.g., dietary changes).

### CROSS-REFERENCE TO RELATED APPLICATIONS

This application claims the benefit of U.S. Application No. 63/562,908, filed on Mar. 8, 2024, which is incorporated herein by reference in its entirety.

### BACKGROUND

Various embodiments relate generally to health care systems, methods, devices and computer programs and, more specifically, relate to intervention-based treatment for chronic medical conditions.

This section is intended to provide a background or context. The description may include concepts that may be pursued, but have not necessarily been previously conceived or pursued. Unless indicated otherwise, what is described in this section is not deemed prior art to the description and claims and is not admitted to be prior art by inclusion in this section.

Chronic medical conditions such as, for example, chronic systemic inflammation, can be a driver of progression to more serious diseases. Thus, interventions which target chronic medical conditions can indirectly improve outcomes for those more serious diseases.

Additionally, computers have changed the way people interact. Digital networks, which may include the use of social media, allow individuals to interact with others online and connect many people with a community. The online community can be utilized to help build positive behaviors and encourage people to make improvements in their lives.

What is needed is a way to use interventions to help provide treatment for chronic medical conditions, such as chronic systemic inflammation, with the aid of computers and online communities.

### SUMMARY

Example aspects of the present disclosure include:

A method to manage progression of a chronic medical condition in an individual according to at least one embodiment of the present disclosure comprises providing a digital communication network (DCN) for members of a population to interact with each other and the DCN; receive a first measure of a biological marker from a first member of the population, wherein the biological marker correlates to the chronic medical condition; receive a second measure of the biological marker from the first member; identify that the first member has an increasing level of the biological marker; analyze a plurality of communications in the DCN between the members of the population; determine at least one communication from a second member of the population that includes an experience of the second member regarding an intervention to decrease the level of the biological marker; and present the at least one communication from the second member to the first member.

Any of the aspects herein, wherein the chronic medical condition is at least one of chronic systemic inflammation (CSI) and prodromal CSI.

Any of the aspects herein, further comprising: receive a third measure of the biological marker from the first member; and identify that the first member has a decreasing level of the biological marker based on the third measure of the biological marker and the second measure of the biological marker.

Any of the aspects herein, further comprising: receive a third measure of the biological marker from the first member; identify that the first member at least one of the same or an increasing level of the biological marker based on the third measure of the biological marker and the second measure of the biological marker; and analyze communications in the DCN between the members of the population; determine at least one communication from a third member of the population that includes an experience of the third member regarding an intervention to decrease the level of the biological marker; and present the at least one communication from the third member to the first member.

Any of the aspects herein, wherein the at least one communication comprises a plurality of communications from a plurality of members, and wherein the method further comprises: prioritizing the plurality of communications based on the level of the biological marker and the information about the first member; and present the plurality of communications to the first member in a prioritized order based on the prioritization.

Any of the aspects herein, wherein a measure of the biological marker is received from an at home sampling kit provided to the first member for testing.

Any of the aspects herein, wherein the at home sampling kit is configured to collect at least one of: blood and saliva.

Any of the aspects herein, wherein the sampling kit is a psychosocial instrument.

Any of the aspects herein, wherein identifying the first member is based on an increase of the level of the biological marker between repeated tests.

Any of the aspects herein, wherein the intervention is a pharmaceutical intervention.

Any of the aspects herein, wherein the pharmaceutical intervention comprises weight loss drugs.

Any of the aspects herein, wherein the intervention is a lifestyle intervention.

Any of the aspects herein, wherein the lifestyle intervention comprises at least one of: fasting, intermittent fasting, stress reduction. improving sleep behaviors, improving mood, reducing anxiety, improving relationships and dietary changes.

Any of the aspects herein, wherein the disease is at least one of: a metabolic disease, such as obesity, diabetes, Cardiovascular disease, Complement Factor H, Chronic Kidney Disease, and Nonalcoholic steatohepatitis; an autoimmune disease, such as, Rheumatoid arthritis, Crohn's disease, eczema, psoriasis, psoriatic arthritis, and Type 1 diabetes; an inflammation moderated lung disease, such as, Chronic obstructive pulmonary disease and Asthma, and an oncological disease.

A method to manage progression of a prodromal chronic systemic inflammation (CSI) related condition according to at least one embodiment of the present disclosure comprises providing a digital communication network (DCN) for members of a population to interact with each other and the DCN; receive a first measure of a biological marker from a first member of the population, wherein the biological marker correlates to the prodromal CSI; receive a second measure of the biological marker from the first member; identify that the first member has an increasing level of the biological marker; analyze a plurality of communications in the DCN between the members of the population; determine at least one communication from a second member of the population that includes an experience of the second member regarding an intervention to decrease the level of the prodromal CSI; and present the at least one communication from the second member to the first member.

Any of the aspects herein, wherein the prodromal CSI related disease is at least one of: prediabetes, metabolic syndrome, hyperinsulinemia, fatty liver disease, and elevated autoantibodies.

A method to manage progression of a chronic medical condition in an individual according to at least one embodiment of the present disclosure comprises receive a first measure of a biological marker from a member of the population, wherein the biological marker correlates to the chronic medical condition; receive a second measure of the biological marker from the member; identify that the member has an increasing level of the biological marker; determine an intervention to decrease the level of the biological marker based on the level of the biological marker and information about the member; and present the intervention to the first member.

Any of the aspects herein, wherein the chronic medical condition is chronic systemic inflammation (CSI).

Any of the aspects herein, further comprising: receive a third measure of the biological marker from the member; and identify that the member has a decreasing level of the biological marker based on the third measure of the biological marker and the second measure of the biological marker.

Any of the aspects herein, further comprising: receive a third measure of the biological marker from the member; receive a fourth measure of the biological marker from the member; identify that the member at least one of the same or an increasing level of the biological marker based on the third measure of the biological marker and the second measure of the biological marker; and determine another intervention to decrease the level of the biological marker based on the level of the biological marker and the information about the member; and present the another intervention to the member.

### BRIEF DESCRIPTION OF THE DRAWINGS

Aspects of the described embodiments are more evident in the following description, when read in conjunction with the attached Figures.

FIG. 1 shows a simplified block diagram of devices in accordance with various embodiments.

FIG. 2 is a logic flow diagram that illustrates a method in accordance with various embodiments.

FIG. 3 is another logic flow diagram that illustrates the operation of a further method in accordance with various embodiments.

FIG. 4A shows an example of a computing system, in accordance with one or more embodiments.

FIG. 4B shows an example of a network, in accordance with one or more embodiments.

### DETAILED DESCRIPTION

Biological markers may be indicative of disease progression and can be used to monitor and/or analyze potential recommendations or interventions to reduce or control the progression of such disease. For example, chronic systemic inflammation (CSI) is typically an indicator of progression to more serious disease. Thus, interventions which target CSI can indirectly improve outcome in those more serious diseases. Such interventions can be determined and delivered to a member of a population in various ways, as described below.

By way of background, as a social process, value can be placed on those members of a community or population that have experience over expertise. Such value can be used to create an atmosphere or community where members can learn about others who have undergone the same interventions that an individual member is contemplating or in the process of doing. Additionally, the community can be used to help support the individual member, which can be a patient under the care of a physician or simply someone interested in the condition and/or disease.

The community can also be used to encourage lifestyle interventions, which are inherently safe. Such community may use the philosophy that any action now is preferred to a “better” action later and also support the concept that ideas and communication are healthcare. Conventionally, lifestyle change has been looked at as in individual pursuit, such as plans personalized just for the individual. Further, medicine is typically a one-on-one activity (reinforced by the privacy concepts the system is based on). In the present disclosure, lifestyle change is seen as highly driven by social parameters and the impact on social parameters is critical, as will be discussed below.

Conventional lifestyle applications may tell individuals the “right thing to do”, which could be right, but given the complexity of the lifestyle change, is likely not to occur. Often, if the lifestyle change suggested works, they can make the individual more dependent on things outside the individual's control. On the other hand, communities share experiences, not expertise, which individuals can try and if they work for them, is a success. In some cases, success can range from slowing the progression of adverse conditions to managing a disease, or the overall risk level in a population.

Lifestyle change may be supported by communities that provide support, ideas, and, in the case of these ideas, access to tools to provide objective data to make meaningful lifestyle changes in an individual user or members of a population. For example, in a community where a person is a peer, the actions they take and learn from are that their volition and may result in increased agency (or autonomy) or self-efficacy. This not only increase the chances of continuous lifestyle improvement, but improved outcomes throughout the heath system.

Thus, it is desirable to provide a community where experience from one or more members can be identified and highlighted for other members based on a given member's increasing level of one or more biological markers. Such community can be implemented in a digital communication network (DCN) where members can interact with each other and the DCN. As one example, the community teaches and encourages members to discover their individual biological marker levels (such as for chronic systemic inflammation (CSI)) and, often more importantly, whether it is increasing or decreasing. The community encourages individuals to share what they are doing and how it impacted their condition. Finally, the members of the community can use these experiences to find things they can try and share.

Further, online communities such as the DCN can be provided so that people can learn about healthy lifestyle practices and work to improve their health between clinical touchpoints, such as office visits. To help incentivize healthy behaviors, individuals can use tools like in-home tests and biosensors that measure how well their health actions are working.

Various embodiments can serve to manage a variety of conditions, such as, CSI. For example, specific inflammatory markers may be of interest, e.g., in diagnosing and approving drugs to treat rheumatoid arthritis (RA). Management of a condition can include maintaining the condition at the present levels, slowing the progression or reversing the condition (if possible). Using the DCN, many members of the population can be assisted which allows the embodiments to be used to manage the health of the population, the health risks of the population and/or reduce health care expense of the population overall.

By way of background, generalized CSI is not specific to any disease and, as such, does not often fit into a single category in a healthcare system. Such characterization results in CSI not being treated in a risk/benefit positive way with drugs or surgery or with any of the tools the provider and insurer community provide. Instead, it is driver and marker of disease progression in general, even if the particular disease is unknown. While the risk-benefit-ratio of drugs and surgery are not positive it can be improved (and therefore disease progression can be slowed) with lifestyle, social situation, emotional management with little or no risk.

Although CSI effects progression of a disease, progression is not typically measured in healthcare. Inflammation and its management impact disease progression and, at some point, disease state. If disease progression can be slowed, the disease state can be avoided and the population is less sick. Thus, it is beneficial to provide methods and systems where members of a population can be identified as having increased levels of biological markers that indicate the start of or progression of a disease and providing interventions such as communications from other members experiencing the same increase levels of the biological markers and how those members improved and decreased their levels of the biological markers.

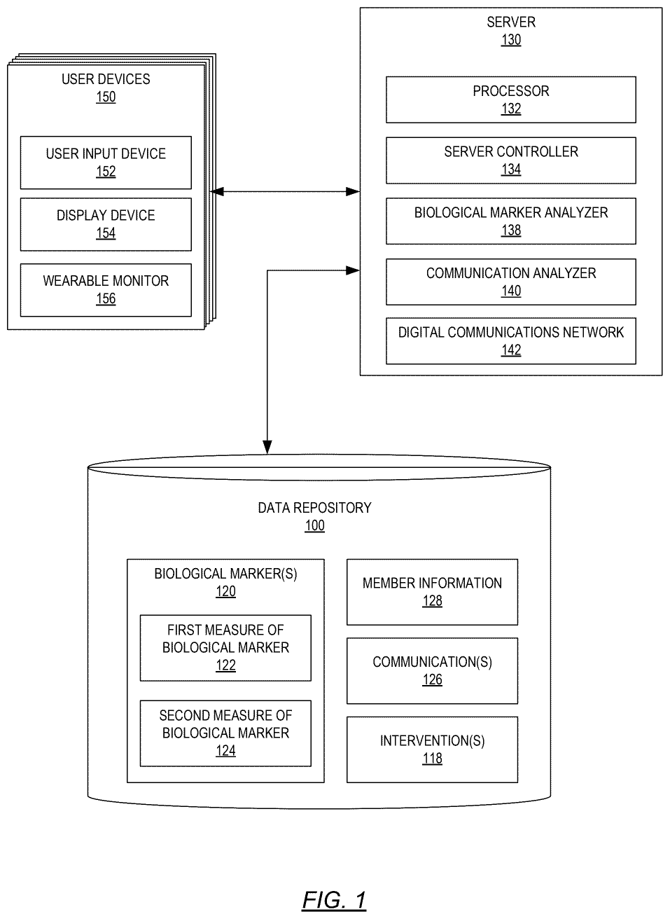

Attention is now turned to the figures. FIG. 1 shows a computing system, in accordance with one or more embodiments. The computing system can be used to, for example, support a digital communication network (DCN) 142 to enable members of a population to communicate with each other. The computing system can also be used to determine level(s) of biological marker(s) (120) of the member(s) through various testing, identify a member that has an increasing level of the biological marker(s) (120), determine an intervention (118) to reduce the level of the biological marker (120) of the member based information about the member and the level of the biological marker(s) (120), analyze and determine communication(s) (126) from other member(s) regarding the intervention (118), and presenting such communication(s) (126) to the member with the increased level of the biological marker (120).

The system shown in FIG. 1 includes a data repository (100). The data repository (100) is a type of storage unit or device (e.g., a file system, database, data structure, or any other storage mechanism) for storing data (described below). The data repository (100) may include multiple different, potentially heterogeneous, storage units and/or devices.

The data repository (100) stores biological markers (120). The biological markers (120) are a measurable characteristic of a biological state or condition or what is happening in an organism or a cell. The biological markers (120) are measured and evaluated using a blood, urine, stool, saliva, breath, or soft tissue sample from a user or member of a population or from heart-rate meters, accelerometer samples, body temperature as well as other signals derived from wearables. The biological markers (120) can also be derived from psychometric instruments and EMA-derived data. The biological markers (120) can be obtained from testing at home or at a clinic. In instances where the biological markers (120) are obtained from at-home tests, the results may be more precise, which can be used to detect benefits that would otherwise be within the variance of a population-controlled sample

The biological markers (120) can be indicative or provide information about, for example, a chronic medical condition. The chronic medical condition may be, for example, chronic systemic inflammation (CSI). More specifically, CSI may be measured based on biological markers (120) of activation of a member's immune system. Other biological markers (120) may include cytokines to antibodies to biomarkers like heart rate variability. Any biological marker (120) may be used and may be specific biological markers (120), general biological markers (120), or may be used as part of an index.

CSI is not specific to any disease, but is rather a driver and marker of disease progression of a disease such as, for example, Crohn's disease, eczema, psoriasis, etc. Thus, monitoring and treating CSI using the biological markers (120) can lead to positive outcomes for a member of the population. For example, an increasing level of the biological markers (120) may indicate an increase in a member's CSI and the corresponding disease. Interventions (118) may be provided to the member or communications (126) from other members who have experience with the intervention (128) may be provided to the member in an effort to reduce the member's biological marker (120) level.

The biological marker (120) may include multiple biological markers (120) obtained from a member at different time periods. For example, the biological marker (120) may include a first measure of a biological marker (122) and a second measure of the biological marker (124) obtained at different times. The difference between the first measure of the biological marker (122) and the second measure of the biological marker (124) can be used to determine if a level of the biological marker (120) is increasing or decreasing. It will be appreciated that the biological marker (120) can have any number of biological markers (120) such as one biological marker (120), two biological markers (120), or more than two biological markers (120).

The data repository (100) also stores member information (128). The member information (128) may include personal information of an individual member that can be used in determining the intervention (118) (described below) for the user such as, height, weight, etc. The member information (128) can include prior medical history, prior interventions (118) and the results of those interventions (118), prior biological markers (120), prior biological marker levels, and any other information provided by the member.

The data repository (100) also stores communications (126). The communications (126) are communications from members in the DCN (142). The communications may be, for example, text-based, image-based, multimedia communications, audio communications, or any combinations thereof. The communications may be between members, communications in a forum, communications with the DCN (142), or communications generated by the DCN (142) (e.g., communications from a chatbot). The communications (126) can include information about a member's experience with a particular intervention (118) and whether the intervention (118) was helpful to the member.

The data repository (100) also stores interventions (118). The intervention (118) is generated for the member based on the level of their biological marker (120) and whether the level has increase, stayed the same, or decreased. The intervention (118) may also be based on the member information (128). With respect to the level of the biological marker (120) if the level of the individual member's biological marker (120) has increased, indicating an increase in, for example, a progression of a disease, then the intervention (118) may provide suggestions to decrease the level of the biological marker (120). Such suggestions may include, for example, lifestyle interventions such as fasting, stress reduction, improving sleep behaviors, improving mood, reducing anxiety, improving relationships, and/or dietary changes. The interventions (118) may also be a medical intervention such as, for example, prescribing medication to the member. In some instances, the intervention (118) may be provided to the individual member's healthcare provider. If the level of the biological marker (120) is the same or decreasing, no intervention (118) may be given or the member may be encouraged to continue their current lifestyle and activities.

In some embodiments, the intervention (118) includes communications (126) from other members who have had the same intervention (118) suggested to them and the member's experience with the intervention (118). For example, the communication (126) may include a member who had an intervention (118) of stress reduction via yoga prescribed to them. The communication (126) may include that the member had a positive result with yoga. On the other hand, the communication (126) may include interventions (118) that did not work for the member. For example, the communication (126) may include that dietary changes did not improve a member's biological marker (120) level for a particular disease or chronic medical condition.

In some embodiments, the intervention (118) may be provided to the population or sub-groups of the population. For example, a sub-group of members may have similar levels of biological markers (120) and the intervention (118) for suggestions on how to decrease their levels of biological markers (120) may be provided to the sub-group.

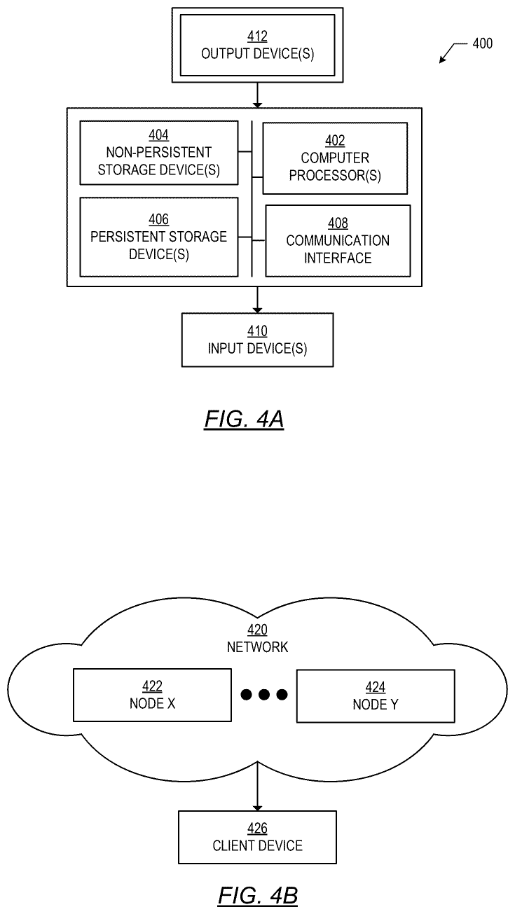

The system shown in FIG. 1 may include other components. For example, the system shown in FIG. 1 also may include a server (130). The server (130) is one or more computer processors, data repositories, communication devices, and supporting hardware and software. The server (130) may be in a distributed computing environment. The server (130) is configured to execute one or more applications, such as a biological marker analyzer (138) or a communication analyzer (140). An example of a computer system and network that may form the server (130) is described with respect to FIG. 4A and FIG. 4B.

The server (130) also includes a computer processor (132). The computer processor (132) is one or more hardware or virtual processors which may execute computer readable program code that defines one or more applications, such as the biological marker analyzer (138) or the communication analyzer (140). An example of the computer processor (132) is described with respect to the computer processor(s) (402) of FIG. 4A.

The server (130) also may include a server controller (134). The server controller (134) is software or application specific hardware which, when executed by the computer processor (402), controls and coordinates operation of the software or application specific hardware described herein. Thus, the sever controller (134) may control and coordinate execution of the biological marker analyzer (138) or the communication analyzer (140).

The server (130) also includes the biological marker analyzer (138). The biological marker analyzer (138) is software or application specific hardware which, when executed by the computer processor (132) provides the level of the biological marker (120). The biological marker analyzer (138) may use the first measure of the biological marker (122), the second measure of the biological marker (124), and any other number of measures of the biological marker (120) to determine the level of the biological marker (120). In some embodiments, the biological marker analyzer (138) may determine a difference in the first measure of the biological marker (122) and the second measure of the biological marker (124) to determine if the level of the biological marker (120) is increasing, decreasing, or has stayed the same.

The server (130) also includes a communication analyzer (140). The communication processor (140) is software or application specific hardware which, when executed by the computer processor (132), determines and/or prioritizes communications (126) in the DCN (142) to present a member. For example, the communication analyzer (140) receives, as input, a level of biological marker (120) of the member and/or the member information (128) and outputs the communication (126) that is relevant to the member. The communication analyzer (140) may also prioritize the communications (126) presented to the member. For example, communications (126) that include interventions (118) that had positive results may be prioritized over communications (126) that include interventions (118) with neutral or negative results. In another example, communications (126) from members that have information related to interventions appropriate for an elevated CSI level, for a disease related to the CSI, or for a disease or prodromal disease related to CSI may be prioritized for a member who has an elevated CSI level as indicated by their biological markers (120). Communications (126) may also be selected or prioritized that include an intervention to change a trajectory of a disease. For example, the intervention (118) may be prescribed medication to improve the member's disease.

The server (130) also includes the digital communications network (DCN) (142). The DCN (142) is a network through which members of a population can interact with each other, or with a system supported by the DCN (142). For example, members can communicate with each other about their chronic medical conditions and interventions that may have worked or did not work for different members. In at least one embodiment, the DCN (142) can also provide access to activity trackers, health testing, tools to improve a member's health, etc.

In some embodiments, the DCN (142) can receive the biological markers (120) from a member or provide instructions for the member to obtain their biological markers (120). For example, if the member uses an at home test to obtain their biological markers (120), the DCN (142) can provide instructions for using the at home test. In other examples, the DCN (142) can provide instructions for visiting a local clinic to obtain their biological markers (120). The DCN (142) can also be used to receive input from the member such as the member information (128). For example, the individual member may use an application on their mobile device to send the member information (128) to the DCN (142) or may wear a wearable device or monitor (156) that can measure and send the member information (128) to the DCN (142).

The DCN (142) can also provide means for members of the population to communicate with each other. Members can share their experience with their interventions (118) and whether an intervention (118) was helpful or not helpful. The DCN (142) can also be used to deliver or transmit the intervention (118) to an individual member. The DCN (142) can also be used by providers and individual members to improve, for example, a member's health by improving the member's lifestyle behaviors. For example, members or providers can pass along information and/or tools that enable a member to easily adopt the healthy action that works best for the member.

The DCN (142) also gives access to information and guidance to help members discover healthy actions that work best for them. Members can use the DCN (142) to, for example, find a community of individuals and other members who can support them and vice versa through online or in-person conversations arranged through the DCN (142).

In the DCN (142), the communications (126) may be prioritized. The communications (126) can be prioritized by, for example, the communication analyzer (140). For example, member messages may be ordered to prioritize messages from members who have had success with their intervention (118). In other examples, the communications (126) can be prioritized based on various factors, such as the individual's own biological information, the individuals they follow in the network, etc. The DCN (142) may also prioritize communications (126) for the member to view which are created by other members who have commented on an intervention (118). These communications (126) between members of the population can be one-to-one, one-to-many, one-to-system, system-to-one, or system-to-many. The system may also feature an AI bot that derives its communications from analysis of communications (126) in the DCN (142) and/or biometrics provided to the DCN (142).

In the DCN (142), members of the population can be identified or self-enrolled based on their shared information. Some members may request information about a class of disease, or a specific disease, and the DCN (142) may follow-up with them to provide information regarding interventions (118) to help manage the class or disease. In some cases, the DCN (142) may use bots to create additional communications (126) to help share relevant information which is in turn also prioritized.

The system shown in FIG. 1 also may include one or more user devices (150). The user devices (150) may be considered remote or local. A remote user device is a device operated by a third-party (e.g., an end user of a chatbot) that does not control or operate the system of FIG. 1. Similarly, the organization that controls the other elements of the system of FIG. 1 may not control or operate the remote user device. Thus, a remote user device may not be considered part of the system of FIG. 1.

In contrast, a local user device is a device operated under the control of the organization that controls the other components of the system of FIG. 1. Thus, a local user device may be considered part of the system of FIG. 1.

In any case, the user devices (150) are computing systems (e.g., the computing system (400) shown in FIG. 4A) that communicate with the server (130). The user devices (150) may include a wearable monitor (156) that can be connected to the DCN (142) and can send information to the DCN (142). In an alternative embodiment, a separate wearable device or monitor (156) may be in communication with the user device (150), such as a smart watch, glucose monitor, or blood pressure monitor. The user devices (150) may also include a user input device (152) and/or a display device (154).

The user devices (150) may also be a member's personal device such as, for example, a computer, a tablet, a smart phone, a cell phone, etc. to interact with the DNC (142). As previously described, the DCN (142) can provide a platform for relaying communications such as public posts and/or direct messages. Additionally, the DCN (142) can provide services and/or applications that can be accessed by the user devices (150).

While FIG. 1 shows a configuration of components, other configurations may be used without departing from the scope of one or more embodiments. For example, various components may be combined to create a single component. As another example, the functionality performed by a single component may be performed by two or more components.

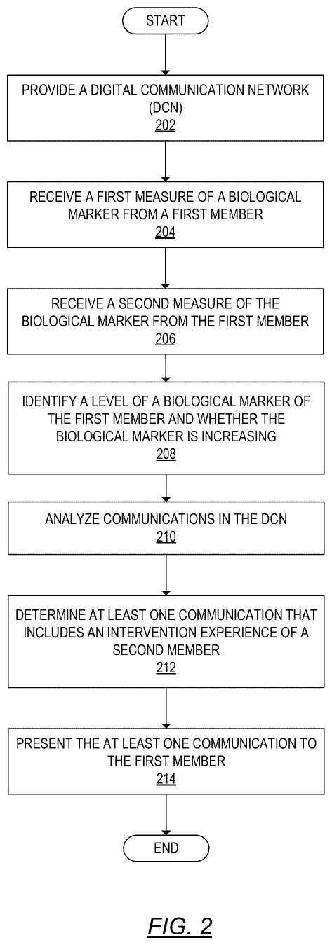

FIG. 2 is a logic flow diagram that illustrates a method, and a result of execution of computer program instructions, in accordance with various embodiments. The method can be used to identify a member of a population that has an increasing level of a biological marker such as, for example, the biological marker (120) and determining at least one communication such as for example, the communication (126) from a DCN such as, for example, the DCN (142) that belongs to another member. The communication can include the other member's experience with an intervention and can be presented to the initial member.

At Step 202, a DCN is provided. The DCN may be the same as or similar to the DCN (142) and provides a network for members of a population to interact with each other or with the DCN (142).

At Step 204, a first measure of a biological marker is received from a first member. The first measure of the biological marker may be the same as or similar to the first measure of the biological marker (122). The first measure of the biological marker may be received from the first member via a user device such as the user device (150). The first measure of the biological marker, as previously described, may be obtained by the first member using an at home test or from a provider at a clinic.

At Step 206, a second measure of the biological marker is received from the first member. The second measure of the biological marker may be the same as or similar to the second measure of the biological marker (124). The second measure of the biological marker can be obtained after the first measure of the biological marker. The second measure of the biological marker can be obtained in the same manner or different manner (e.g., by an at home test or at a clinic) as the first measure of the biological marker.

It will be appreciated that in some embodiments, the method includes receiving more than one biological marker such as a third measure of the biological marker, a fourth measure of the biological marker, etc.

At Step 208, a level of a biological marker of the first member is identified and whether the biological marker is increasing. The level of the biological marker can be obtained from, for example, a biological marker analyzer such as the biological marker analyzer 138. The biological marker analyzer can receive the first measure of the biological marker and the second measure of the biological marker as input, and output the level of the biological marker. In some instances, the level of the biological marker is equal to a difference between the first measure of the biological marker and the second measure of the biological marker. In instances where the first member is identified as having an increasing biological marker level, the method may proceed to the remaining steps.

In some embodiments, the first member may be additionally identified by identifying that the member is on a spectrum of a disease. For example, the first member may be identified by an increase level of CSI as indicated by the increased level of the biological marker and also that the first member has early symptoms of psoriasis.

At Step 210, communications in the DCN are analyzed. The communications may be analyzed by, for example, a communication analyzer such as the communication analyzer (140). The communication analyzer receives at least the level of the biological marker of the first member as an input and in some embodiments, also receives member information such as the member information (128) to analyze communications from other members in the DCN. For example, the member information may be used to identify communications from another member with similar medical health conditions. In another example, the level of the biological marker may be used to identify communications from another member with the same or similar level of the biological marker.

At Step 212, at least one communication is determined based on the analysis that includes an intervention experience of a second member. The intervention may be the same as or similar to the intervention (118). As previously described, the intervention is a suggestion or prescribed activity or prescription to a member to aid in decreasing the level of the biological marker of the member. The at least one communication thus includes the second member's experience with their prescribed intervention that may be useful to the first member to reduce the first member's biological marker level.

At Step 214, the at least one communication is presented to the first member. The communication may be presented to the first member by the DCN via the user device. For example, the at least one communication may be presented to the user as a recommended communication or a direct message to the user by a graphical user interface (GUI) of the DCN. In other embodiments, the at least one communication may be transmitted to a provider of the first member with instructions to present the at least one communication to the first member by the provider.

The method of FIG. 2 described above may have more or less steps than shown. Further, steps may be repeated as needed. For example, the Steps 204, 206, and 206 may be repeated to evaluate if the communication provided was effective.

In some embodiments, the method may also include receiving a third measure of the biological marker from the first member. The third measure of the biological marker can be used with the second measure of the biological marker to determine if the first member's biological marker level has increased or decreased. In instances where the biological marker level has decreased, the method may end or the method may include providing recommendations to continue intervention(s) that the first member has been participating in. In instances where the biological marker level has increased, the method may continue to repeat the Steps 210, 212, and 214 to identify additional communications to present to the first member.

The method may also include prioritizing the communications based on the level of the biological marker and the information about the first member and to present the plurality of communications to the first member in a prioritized order based on the prioritization. As previously described, the communication analyzer may prioritize the communications presented to the member. For example, communications that include interventions that had positive results may be prioritized over communications that include interventions with neutral or negative results. In another example, communications from members that have information related to interventions appropriate for an elevated CSI level, for a disease related to the CSI, or for a disease or prodromal disease related to CSI may be prioritized for a member who has an elevated CSI level as indicated by their biological markers. Communications may also be selected or prioritized that include an intervention to change a trajectory of a disease. For example, the intervention may be prescribed medication to improve the member's disease.

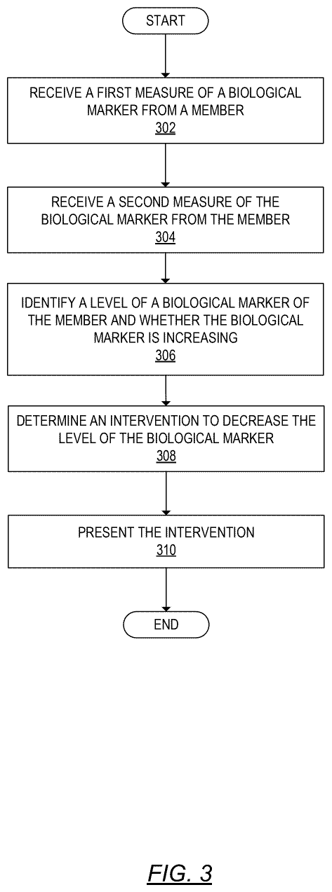

FIG. 3 is a logic flow diagram that illustrates a method, and a result of execution of computer program instructions, in accordance with various embodiments. The method can be used to determine a member of a population that has an increasing level of a biological marker and determining an intervention for the member to decrease the level of the biological marker.

At Step 302, a first measure of a biological marker is received from a member. The Step 302 is the same as or similar to the Step 204 of FIG. 2 above.

At Step 304, a second measure of the biological marker is received from the member. The Step 304 is the same as or similar to the Step 206 of FIG. 2 above.

At Step 306, identifying a level of a biological marker of the member and whether the biological marker is increasing. The Step 306 is the same as or similar to the Step 208 of FIG. 2 above.

At Step 308, an intervention to decrease the level of the biological marker is determined.

At Step 310, the intervention is presented. The intervention may be the same as or similar to the intervention (118). The intervention, as previously described, is generated for the member based on the level of their biological marker and whether the level has increase, stayed the same, or decreased. For example, if the level of the individual member's biological marker has increased, indicating an increase in, for example, a progression of a disease, then the intervention may provide suggestions to decrease the level of the biological marker. Such suggestions may include, for example, lifestyle interventions such as fasting, stress reduction, improving sleep behaviors, improving mood, reducing anxiety, improving relationships, and/or dietary changes. The interventions may also be a medical intervention such as, for example, prescribing medication to the member. In some instances, the intervention may be provided to the individual member's healthcare provider so that the healthcare provider can deliver the intervention or information about the intervention to the member.

The method of FIG. 3 described above may have more or less steps than shown. Further, steps may be repeated as needed. For example, the Steps 302, 304, and 306 may be repeated to determine if the intervention was effective.

A specific example of the above methods of FIGS. 2 and 3 will now be described. In such example, an individual member of a population may sign up as a member of a DCN to aid in achieving, for example, a healthy lifestyle or improve a chronic medical condition that the individual member is diagnosed with. For example, the individual member may have Crohn's disease. The individual member then may receive instructions or a prompt from the DCN to determine their biological marker level as an indicator of the level or severity of their Crohn's disease. The biological marker that the individual member tests for may be chronic systemic inflammation (CSI).

In the same example, the individual user may receive at-home tests to obtain their biological markers. The individual may take one test and upload or otherwise send the results to the DCN. After a certain time such as a month or several months, the user may take another test and upload or otherwise send the results to the DCN. The DCN (or any server) can then determine if the biological marker level of the individual user is increasing, decreasing, or staying the same. If the biological marker level is decreasing or staying the same, then the DCN may suggest that the individual user continue with their daily life as this indicates that the CSI—and thus the correlating Crohn's disease—is stable.

In instances where the level of the biological marker is increasing, this may indicate that the CSI is increasing, and thus, the correlating Crohn's disease may be advancing. In such instances, the DCN may then determine and provide an intervention for the individual member based on the level of biological marker—and in some cases, information about the individual member—to decrease the level of biological marker. Such intervention may be, for example, dietary changes. Alternatively, the DCN or communication analyzer may analyze communications from other members that have also made dietary changes in order to decrease their respective levels of the biological marker that may be relevant to the individual user. The DCN may the present these communications to the individual member with or without the intervention. The communications may provide support to the individual member in that the individual member can see that other members have experienced the same level of biological marker and were successful in reducing their respective levels of the biological marker.

One or more embodiments may be implemented on a computing system specifically designed to achieve an improved technological result. When implemented in a computing system, the features and elements of the disclosure provide a significant technological advancement over computing systems that do not implement the features and elements of the disclosure. Any combination of mobile, desktop, server, router, switch, embedded device, or other types of hardware may be improved by including the features and elements described in the disclosure.

For example, as shown in FIG. 4A, the computing system (400) may include one or more computer processor(s) (402), non-persistent storage device(s) (404), persistent storage device(s) (406), a communication interface (408) (e.g., Bluetooth interface, infrared interface, network interface, optical interface, etc.), and numerous other elements and functionalities that implement the features and elements of the disclosure. The computer processor(s) (402) may be an integrated circuit for processing instructions. The computer processor(s) (402) may be one or more cores, or micro-cores, of a processor. The computer processor(s) (402) includes one or more processors. The computer processor(s) (402) may include a central processing unit (CPU), a graphics processing unit (GPU), a tensor processing unit (TPU), combinations thereof, etc.

The input device(s) (410) may include a touchscreen, keyboard, mouse, microphone, touchpad, electronic pen, or any other type of input device. The input device(s) (410) may receive inputs from a user that are responsive to data and messages presented by the output device(s) (412). The inputs may include text input, audio input, video input, etc., which may be processed and transmitted by the computing system (400) in accordance with one or more embodiments. The communication interface (408) may include an integrated circuit for connecting the computing system (400) to a network (not shown) (e.g., a local area network (LAN), a wide area network (WAN) such as the Internet, mobile network, or any other type of network) or to another device, such as another computing device, and combinations thereof.

Further, the output device(s) (412) may include a display device, a printer, external storage, or any other output device. One or more of the output device(s) (412) may be the same or different from the input device(s) (410). The input device(s) (410) and output device(s) (412) may be locally or remotely connected to the computer processor(s) (402). Many different types of computing systems exist, and the aforementioned input device(s) (410) and output device(s) (412) may take other forms. The output device(s) (412) may display data and messages that are transmitted and received by the computing system (400). The data and messages may include text, audio, video, etc., and include the data and messages described above in the other figures of the disclosure.

Software instructions in the form of computer readable program code to perform embodiments may be stored, in whole or in part, temporarily or permanently, on a non-transitory computer readable medium such as a solid-state drive (SSD), compact disk (CD), digital video disk (DVD), storage device, a diskette, a tape, flash memory, physical memory, or any other computer readable storage medium. Specifically, the software instructions may correspond to computer readable program code that, when executed by the computer processor(s) (402), is configured to perform one or more embodiments, which may include transmitting, receiving, presenting, and displaying data and messages described in the other figures of the disclosure.

The computing system (400) in FIG. 4A may be connected to, or be a part of, a network. For example, as shown in FIG. 4B, the network (420) may include multiple nodes (e.g., node X (422) and node Y (424), as well as extant intervening nodes between node X (422) and node Y (424)). Each node may correspond to a computing system, such as the computing system shown in FIG. 4A, or a group of nodes combined may correspond to the computing system shown in FIG. 4A. By way of an example, embodiments may be implemented on a node of a distributed system that is connected to other nodes. By way of another example, embodiments may be implemented on a distributed computing system having multiple nodes, where each portion may be located on a different node within the distributed computing system. Further, one or more elements of the aforementioned computing system (400) may be located at a remote location and connected to the other elements over a network.

The nodes (e.g., node X (422) and node Y (424)) in the network (420) may be configured to provide services for a client device (426). The services may include receiving requests and transmitting responses to the client device (426). For example, the nodes may be part of a cloud computing system. The client device (426) may be a computing system, such as the computing system shown in FIG. 4A. Further, the client device (426) may include or perform all or a portion of one or more embodiments.

The computing system of FIG. 4A may include functionality to present data (including raw data, processed data, and combinations thereof) such as results of comparisons and other processing. For example, presenting data may be accomplished through various presenting methods. Specifically, data may be presented by being displayed in a user interface, transmitted to a different computing system, and stored. The user interface may include a graphical user interface (GUI) that displays information on a display device. The GUI may include various GUI widgets that organize what data is shown, as well as how data is presented to a user.

Furthermore, the GUI may present data directly to the user, e.g., data presented as actual data values through text, or rendered by the computing device into a visual representation of the data, such as through visualizing a data model.

Various operations described are purely exemplary and imply no particular order. Further, the operations can be used in any sequence when appropriate and can be partially used. With the above embodiments in mind, it should be understood that additional embodiments can employ various computer-implemented operations involving data transferred or stored in computer systems. These operations are those requiring physical manipulation of physical quantities. Usually, though not necessarily, these quantities take the form of electrical, magnetic, or optical signals capable of being stored, transferred, combined, compared, and otherwise manipulated.

Any of the operations described that form part of the presently disclosed embodiments may be useful machine operations. Various embodiments also relate to a device or an apparatus for performing these operations. The apparatus can be specially constructed for the required purpose, or the apparatus can be a general-purpose computer selectively activated or configured by a computer program stored in the computer. In particular, various general-purpose machines employing one or more processors coupled to one or more computer readable medium, described below, can be used with computer programs written in accordance with the teachings herein, or it may be more convenient to construct a more specialized apparatus to perform the required operations.

The procedures, processes, and/or modules described herein may be implemented in hardware, software, embodied as a computer-readable medium having program instructions, firmware, or a combination thereof. For example, the functions described herein may be performed by a processor executing program instructions out of a memory or other storage device.

The foregoing description has been directed to particular embodiments. However, other variations and modifications may be made to the described embodiments, with the attainment of some or all of their advantages. Modifications to the above-described systems and methods may be made without departing from the concepts disclosed herein. Accordingly, the invention should not be viewed as limited by the disclosed embodiments. Furthermore, various features of the described embodiments may be used without the corresponding use of other features. Thus, this description should be read as merely illustrative of various principles, and not in limitation of the invention.

What is claimed is:

1. A method to manage progression of a chronic medical condition in an individual, the method comprising:
providing a digital communication network (DCN) for members of a population to interact with each other and the DCN;
receive a first measure of a biological marker from a first member of the population, wherein the biological marker correlates to the chronic medical condition;
receive a second measure of the biological marker from the first member;
identify that the first member has an increasing level of the biological marker;
analyze a plurality of communications in the DCN between the members of the population;
determine at least one communication from a second member of the population that includes an experience of the second member regarding an intervention to decrease the level of the biological marker; and
present the at least one communication from the second member to the first member.

2. The method of claim 1, wherein the chronic medical condition is at least one of chronic systemic inflammation (CSI) and prodromal CSI.

3. The method of claim 1, further comprising:
receive a third measure of the biological marker from the first member; and
identify that the first member has a decreasing level of the biological marker based on the third measure of the biological marker and the second measure of the biological marker.

4. The method of claim 1, further comprising:
receive a third measure of the biological marker from the first member;
identify that the first member at least one of the same or an increasing level of the biological marker based on the third measure of the biological marker and the second measure of the biological marker; and
analyze communications in the DCN between the members of the population;
determine at least one communication from a third member of the population that includes an experience of the third member regarding an intervention to decrease the level of the biological marker; and
present the at least one communication from the third member to the first member.

5. The method of claim 1, wherein the at least one communication comprises a plurality of communications from a plurality of members, and wherein the method further comprises:
prioritizing the plurality of communications based on the level of the biological marker and the information about the first member; and
present the plurality of communications to the first member in a prioritized order based on the prioritization.

6. The method of claim 1, wherein a measure of the biological marker is received from an at home sampling kit provided to the first member for testing.

7. The method of claim 6, wherein the at home sampling kit is configured to collect at least one of: blood and saliva.

8. The method of claim 6, wherein the sampling kit is a psychosocial instrument.

9. The method of claim 4, wherein identifying the first member is based on an increase of the level of the biological marker between repeated tests.

10. The method of claim 1, wherein the intervention is a pharmaceutical intervention.

11. The method of claim 10, wherein the pharmaceutical intervention comprises weight loss drugs.

12. The method of claim 1, wherein the intervention is a lifestyle intervention.

13. The method of claim 12, wherein the lifestyle intervention comprises at least one of: fasting, intermittent fasting, stress reduction. improving sleep behaviors, improving mood, reducing anxiety, improving relationships and dietary changes.

14. The method of claim 1, wherein the disease is at least one of:
a metabolic disease, such as obesity, diabetes, Cardiovascular disease, Complement Factor H, Chronic Kidney Disease, and Nonalcoholic steatohepatitis;
an autoimmune disease, such as, Rheumatoid arthritis, Crohn's disease, eczema, psoriasis, psoriatic arthritis, and Type 1 diabetes;
an inflammation moderated lung disease, such as, Chronic obstructive pulmonary disease and Asthma, and
an oncological disease.

15. A method to manage progression of a prodromal chronic systemic inflammation (CSI) related condition, the method comprising:
providing a digital communication network (DCN) for members of a population to interact with each other and the DCN;
receive a first measure of a biological marker from a first member of the population, wherein the biological marker correlates to the prodromal CSI;
receive a second measure of the biological marker from the first member;
identify that the first member has an increasing level of the biological marker;
analyze a plurality of communications in the DCN between the members of the population;
determine at least one communication from a second member of the population that includes an experience of the second member regarding an intervention to decrease the level of the prodromal CSI; and
present the at least one communication from the second member to the first member.

16. The method of claim 15, wherein the prodromal CSI related disease is at least one of:
prediabetes, metabolic syndrome, hyperinsulinemia, fatty liver disease, and elevated autoantibodies.

17. A method to manage progression of a chronic medical condition in an individual, the method comprising:
receive a first measure of a biological marker from a member of the population, wherein the biological marker correlates to the chronic medical condition;
receive a second measure of the biological marker from the member;
identify that the member has an increasing level of the biological marker;
determine an intervention to decrease the level of the biological marker based on the level of the biological marker and information about the member; and
present the intervention to the first member.

18. The method of claim 17, wherein the chronic medical condition is chronic systemic inflammation (CSI).

19. The method of claim 17, further comprising:
receive a third measure of the biological marker from the member; and
identify that the member has a decreasing level of the biological marker based on the third measure of the biological marker and the second measure of the biological marker.

20. The method of claim 17, further comprising:
receive a third measure of the biological marker from the member;
receive a fourth measure of the biological marker from the member;
identify that the member at least one of the same or an increasing level of the biological marker based on the third measure of the biological marker and the second measure of the biological marker; and
determine another intervention to decrease the level of the biological marker based on the level of the biological marker and the information about the member; and
present the another intervention to the member.
